(19) United States
(12) Patent Application Publication
(10) Pub. No.: US 2023/0389821 A1
(43) Pub. Date: Dec. 7, 2023

(54) ESTIMATING VERTICAL OSCILLATION AT WRIST

(71) Applicant: Apple Inc., Cupertino, CA (US)

(72) Inventors: Richard A. Fineman, Campbell, CA (US); Adeeti V. Ullal, Emerald Hills, CA (US); Allison L. Gilmore, Redwood City, CA (US); Gabriel A. Blanco, San Francisco, CA (US); Karthik Jayaraman Raghuram, Foster City, CA (US); Mark P. Sena, Larkspur, CA (US); Maryam Etezadi-Amoli, Santa Clara, CA (US); James J. Dunne, San Francisco, CA (US); Po An Lin, San Jose, CA (US)

(21) Appl. No.: 18/205,476

(22) Filed: Jun. 2, 2023

Related U.S. Application Data
(60) Provisional application No. 63/349,089, filed on Jun. 4, 2022.

(51) Int. Cl. A61B 5/11; A61B 5/00

(52) U.S. Cl. CPC A61B 5/1114; A61B 5/681; A61B 5/7264; A61B 2562/0219

(57) ABSTRACT

Enclosed are embodiments for estimating vertical oscillation (VO) at the wrist. In some embodiments, a method comprises: obtaining, with a wearable device worn on a wrist of a user, sensor data indicative of the user's acceleration and rotation rate; estimating centripetal acceleration based on the user's acceleration and rotation rate; calculating a modified user's acceleration by subtracting the estimated centripetal acceleration from the user's acceleration; estimating center of mass (CoM) acceleration by decoupling an arm swing component of the user's acceleration from the modified user's acceleration; and computing vertical oscillation of the user's CoM using a machine learning model with at least the CoM acceleration as input to the machine learning model, or by integrating vertical acceleration derived from the CoM acceleration and a gravity vector.

### CROSS-REFERENCE TO RELATED APPLICATION

This application claims priority to U.S. Provisional Patent Application No. 63/349,089, filed Jun. 4, 2022, the entire contents of which are incorporated herein by reference.

### TECHNICAL FIELD

This disclosure relates generally to health monitoring and fitness applications.

### BACKGROUND

Vertical oscillation (VO) is the amount that the torso of a runner moves vertically with each step while running. Running coaches believe that lower vertical oscillation is more economical because less energy is wasted bouncing up and down. Most runners oscillate somewhere between 6 to 13 cm. To calculate VO, some existing methods require a sensor (e.g., accelerometer) located near the center of mass (CoM) of the runner, such as attached to the runner's torso or waist. Other existing methods require a sensor to be attached to the runner's foot. Although the purchase of these sensors may be justifiable for a professional athlete in training, the average runner may only have a wrist worn smartwatch or fitness band, and may not want to purchase additional wearable devices that are limited to fitness applications.

### SUMMARY

Embodiments are disclosed for estimating VO at the wrist. In some embodiments, a method comprises: obtaining, with at least one processor of a wearable device worn on a wrist of a user, sensor data indicative of the user's acceleration and rotation rate; estimating, with the at least one processor, centripetal acceleration based on the user's acceleration and rotation rate; calculating, with the at least one processor, a modified user's acceleration by subtracting the estimated centripetal acceleration from the user's acceleration; estimating, with the at least one processor, center of mass (CoM) acceleration by decoupling an arm swing component of the user's acceleration from the modified user's acceleration; and computing, with the at least one processor, vertical oscillation of the user's CoM using a machine learning model with at least the CoM acceleration as input to the machine learning model.

In some embodiments, estimating centripetal acceleration comprises: determining a primary rotational axis based on the user's rotation rate; estimating a tangential acceleration based on the user's acceleration and cadence; estimating tangential velocity by integrating the tangential acceleration; calculating a magnitude of the arm swing rotation rate about the primary rotational axis; and estimating the centripetal acceleration as a cross-product of the magnitude of the arm swing rotation rate and the tangential velocity.

In some embodiments, the method further comprises: computing, with the at least one processor, a vertical oscillation of the user's CoM using the machine learning model with the CoM acceleration, tangential acceleration, estimated centripetal acceleration, user acceleration, primary rotational axis and user rotation rate as inputs to the machine learning model.

In some embodiments, the primary rotational axis is determined using principal component analysis (PCA).

In some embodiments, estimating a tangential acceleration includes filtering the tangential acceleration from the user's acceleration based on cadence of the user to generate a modified user acceleration.

In some embodiments, decoupling the arm swing component of the user's acceleration from the modified user's acceleration includes filtering the modified user's acceleration based on a step frequency of the user.

In some embodiments, the machine learning model is a random forest that outputs an estimate of vertical oscillation at the CoM per stride.

In some embodiments, a method comprises: obtaining, with at least one processor of a wearable device worn on a wrist of a user, sensor data indicative of the user's acceleration and rotation rate; estimating, with the at least one processor, centripetal acceleration based on the user's acceleration and rotation rate; calculating, with the at least one processor, a modified user's acceleration by subtracting the estimated centripetal acceleration from the user's acceleration; estimating, with the at least one processor, center of mass (CoM) acceleration by decoupling an arm swing component of the user's acceleration from the modified user's acceleration; determining, with the at least one processor, a gravity vector based on the sensor data; determining, with the at least one processor, vertical acceleration by projecting the CoM acceleration onto the gravity vector; integrating, with the at least one processor, the vertical acceleration to get vertical velocity; integrating, with the at least one processor, the vertical velocity to get vertical position; computing, with the at least one processor, vertical oscillation as the difference between maximum and minimum vertical position per step.

In some embodiments, a system comprises: at least one processor; memory storing instructions that when executed by the at least one processor, cause the at least one processor to perform any of the preceding methods recited above.

In some embodiments, a non-transitory, computer-readable storage medium having stored thereon instructions that when executed by the at least one processor, causes the at least one processor to perform any of the preceding methods recited above.

Other embodiments can include an apparatus, computing device and non-transitory, computer-readable storage medium.

Particular embodiments described herein provide one or more of the following advantages. VO can be determined by a single wearable device (e.g., smartwatch) attached to the wrist, thus avoiding the inconvenience and cost of purchasing devices that are worn at the torso/waist or foot.

The details of one or more implementations of the subject matter are set forth in the accompanying drawings and the description below. Other features, aspects and advantages of the subject matter will become apparent from the description, the drawings and the claims.

### BRIEF DESCRIPTION OF THE DRAWINGS

FIG. 1 illustrates running form metrics at the wrist, according to some embodiments.

FIG. 2 illustrates running form metrics from feet to wrist, according to some embodiments.

FIG. 3A is a block diagram of a system for estimating VO that combines mechanics-based feature engineering with machine learning, according to some embodiments.

FIG. 3B is a block diagram of the system of FIG. 3A but using integration to obtain VO, according to some embodiments.

FIG. 4 illustrates modeling upper body motion, according to some embodiments.

FIG. 5 is flow diagram of a process of estimating VO at the wrist, according to some embodiments.

FIG. 6 is example system architecture implementing the features and operations described in reference to FIGS. 1-5.

### DETAILED DESCRIPTION

### Example System

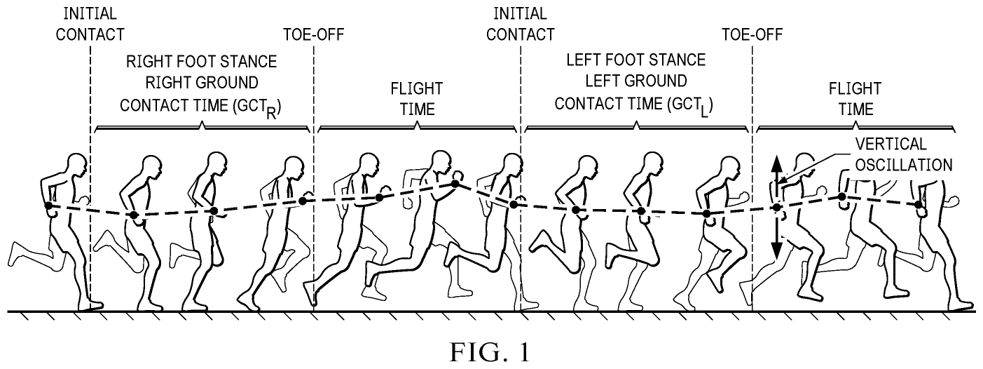

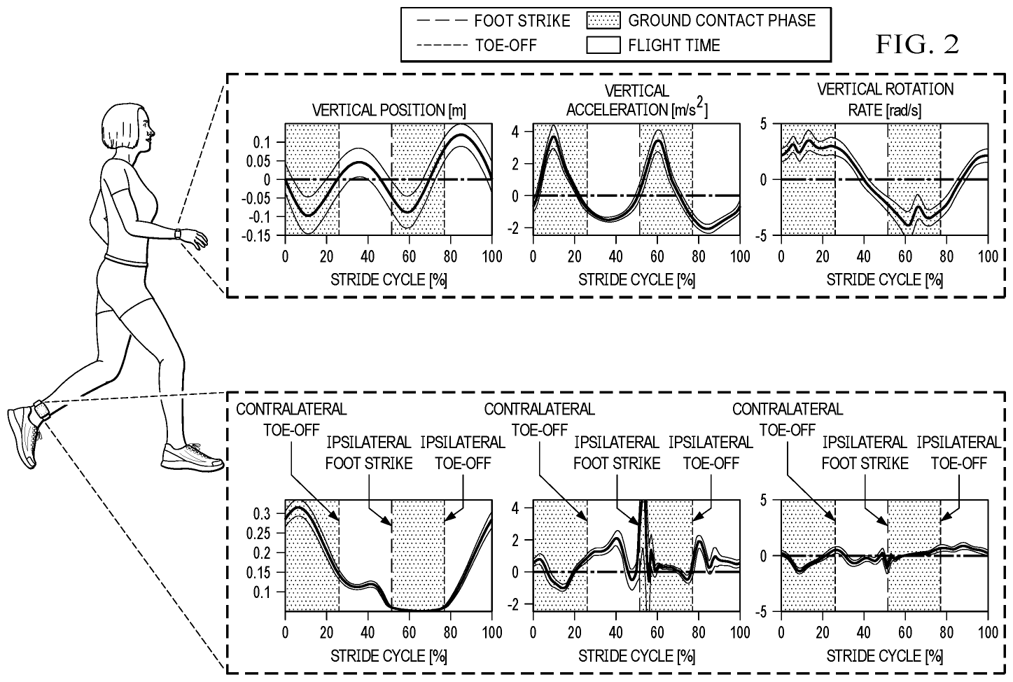

FIG. 1 illustrates VO over a stride cycle that includes a right foot stance, followed by a first flight time, followed by a left foot stance, followed by a second flight time¹. If the runner is wearing a sensor at the waist or torso (e.g., an accelerometer) the vertical oscillation can be measured closer to the CoM. If, however, the runner is wearing a single sensor on their wrist, vertical oscillation cannot be measured directly due to biomechanical linkage from the torso to the wrist. Although one could attempt to model the biomechanics with a biomechanical linkage model, such a model can be complex and will not account for population diversity. ¹ FIGS. 1 and 2 adapted from Uchida, Thomas K. et. al. Biomechanics of Movement—The Science of Sports, Robotics, and Rehabilitation. MIT Press Ltd., United States, 2021.

FIG. 2 illustrates running dynamics at the feet and the wrist, according to some embodiments. The upper plots from left to right are vertical position (m), vertical acceleration (m/s²) and vertical rotation rate (rad/s) measured at the wrist, respectively, versus a percentage of stride cycle. The lower plots from left to right are the same but measured at the feet. The vertical dashed lines separate the different stride events: foot strike, toe-off, ground contact phase and flight phase.

As can be observed from the plots, vertical position, acceleration and rotation rate dynamics at the feet are observable at the wrist as peaks and step periodicity (for acceleration) and stride periodicity (for rotation rate) with a phase shift from the gait event time of interest. Thus, the vertical position, acceleration and rotation rate measured at the wrist can be used to estimate (infer) gait event times using ML models.

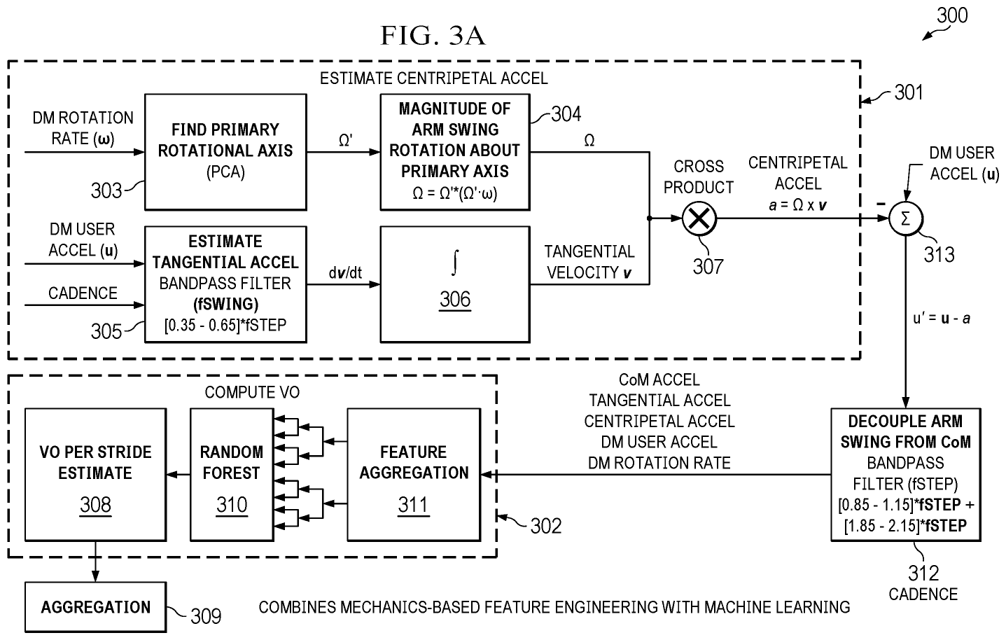

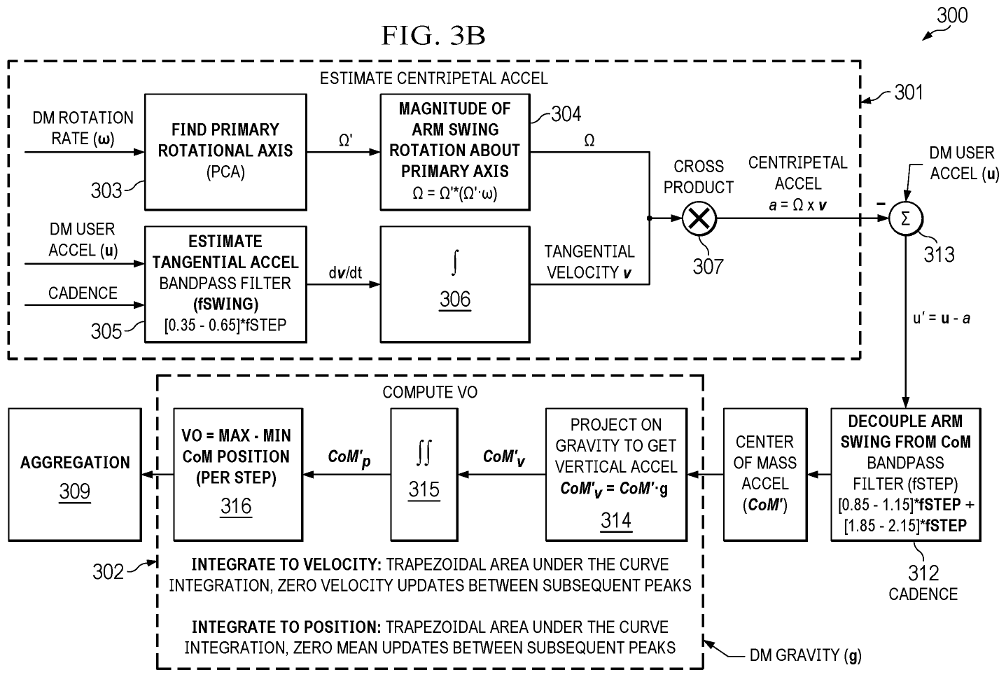

FIG. 3A is a block diagram of ML model 300 for estimating VO that combines mechanics-based feature engineering with machine learning, according to some embodiments. In the example shown, ML model 300 includes a centripetal acceleration estimator 301 and VO estimator 302. Centripetal acceleration estimator 301 receives a window of sensor data including device motion (DM) rotation rate vector ω and user acceleration vector u in body coordinates. The acceleration vector and rotation rate vector can be generated by, for example, 3-axis accelerometers and 3-axis gyros embedded in a wrist worn wearable device, such as a smartwatch. The window of sensor data is selected to include at least one stride cycle.

The rotation rate vector ω is input into principal component analysis (PCA) unit 303 which estimates a primary rotation axis vector, Ω′. The rotation rate 304 about the primary rotational axis is computed according to Equation [1].

Ω=Ω′*(Ω′·ω). [1]

Tangential acceleration estimator 305 receives DM user acceleration u and step frequency/cadence (e.g., form a digital pedometer) and estimates tangential acceleration (dv/dt), which is integrated 306 to give tangential/transverse velocity v.

The centripetal acceleration a is calculated from the cross-product 307 of Ω and v, as shown in Equation [2]:

a=Ω×v. [2]

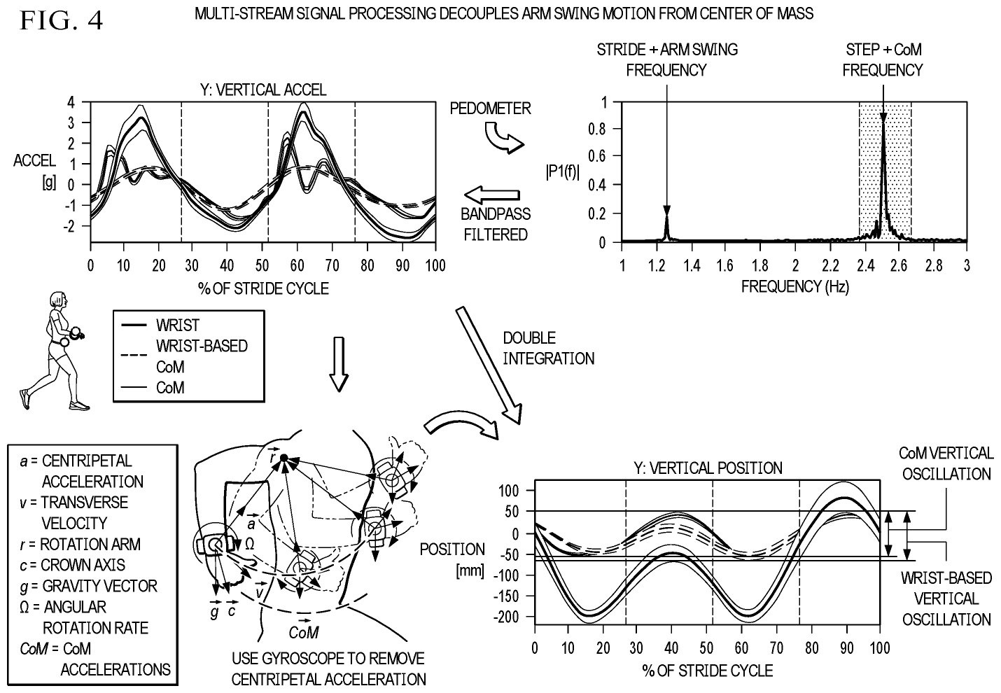

The centripetal acceleration a is then subtracted 313 from the user acceleration u to give a modified user acceleration a′=u−a with the estimated centripetal acceleration removed. This vector is input into arm swing decoupler 312, which applies a bandpass filter to the input to decouple the arm swing acceleration component from the modified user acceleration, as described in reference to FIG. 4. The output of arm swing decoupler 312 is the CoM acceleration with the arm swing acceleration component removed. The CoM acceleration, tangential acceleration, centripetal acceleration, DM user acceleration and DM rotation rate are input features into VO estimator 302.

In some embodiments, VO estimator 302 includes feature aggregator 311 and random forest model 310. Feature aggregator windows data (e.g., per arm swing, features separating forward and backward parts of arm swing) and aggregating the data over time in various ways (minimum, maximum, mean, etc.) for random forest 310. The output of random forest model 310 is a VO per stride estimate, which can be made available to variety of applications through, for example, an application programming interface (API).

In some embodiments, base signals and feature used by random forest model 310 and the sources that the base signals and features are derived from are listed in Tables I and II.

TABLE I
Base Signals/Derived From

| Base Signal | Derived From |
|---|---|
| User acceleration | Device motion (for inertial frame) |
| Gyro rotation rate | Device motion (for inertial frame) |
| Tangential acceleration | Arm swing − CoM decoupling |
| Centripetal acceleration | Arm swing − CoM decoupling |
| Tangential velocity | Arm swing − CoM decoupling |
| COM acceleration | Arm swing − CoM decoupling |
| Arm swing rotation axis | Arm swing − CoM decoupling |
| (output from PCA) | |

TABLE II
Features/Derived From

| Feature | Derived From |
|---|---|
| Height | User entry |
| Weight | User entry |
| BMI | User entry |
| Gender | User entry |
| Cadence | Pedometer |
| Cadence | Arm swing extrema |
| Device wrist | User entry |
| Crown orientation | User entry |

In some embodiments, each base signal in Table I creates 8 derived signals (3 device frame components, 3 inertial frame components, L2 norm of non-vertical signal, L2 norm of entire signal). Additionally, each derived signal is aggregated in 6 ways (max, min, range, mean, standard deviation, area under the curve) over periods demarcated by arm swing extrema. The arm swing extrema is identified using peak to peak detections from the tangential acceleration derived from arm swing—CoM decoupling. In some embodiments, the feature set input into random forest model 310 includes aggregations over a full arm swing (backward and forward), forward swing only and backward swing only.

In some embodiments, VO estimator 302 can use other machine learning models, including one or more of deep learning networks, support vector machines (SVMs), Extreme Gradient Boosting (XGboost), Adaptive Boosting (AdaBoost), LightGBM, CatBoost and the like.

FIG. 3B is a block diagram of the system of FIG. 3A using integration to obtain VO, according to some embodiments. In this embodiment, the CoM acceleration computed in FIG. 3A is projected onto the gravity vector to get vertical acceleration. The vertical acceleration is integrated twice to get vertical position. For example, the trapezoidal area under the curve integration can be used with zero velocity updates between subsequent peaks. VO is calculated as the difference between maximum and minimum vertical positions per step.

FIG. 4 illustrates modeling of upper body motion, according to some embodiments. The bottom-left plot illustrates upper body motion during a stride cycle, and in particular the use of the gyro sensor at the wrist to remove centripetal acceleration as described in reference to FIG. 3. The vector quantities at the wrist include centripetal acceleration a, tangential/transverse velocity v, rotation lever arm r, crown axis c (e.g., unit vector normal to the crown of a smartwatch), gravity vector g and angular rotation rate Ω about the primary rotation axis. Also shown is the CoM of the runner. For wearable devices that do not have crown (e.g., fitness band), c can be replaced with another suitable vector in any desired device right-handed coordinate frame to define orientation of the wearable device at the wrist.

The top-left plot illustrates accelerations at the wrist, wrist-based CoM and the CoM, where wrist-based CoM is computed as shown in FIG. 3 to remove arm swing motion. These accelerations are bandpass filtered to remove the stride plus arm swing frequency as illustrated in the top-right frequency spectrum plot, where the shaded portion is the passband of the filter. In some embodiments, the accelerations are integrated twice to give vertical position at the wrist, wrist-based CoM and the CoM, which are illustrated in the bottom-right plot. As can be observed from the bottom-right plot, the wrist-based position computed according to FIG. 3 closely follows the CoM position across the stride cycle. Accordingly, the wrist-based VO closely follows the CoM VO as desired.

### Example Processes

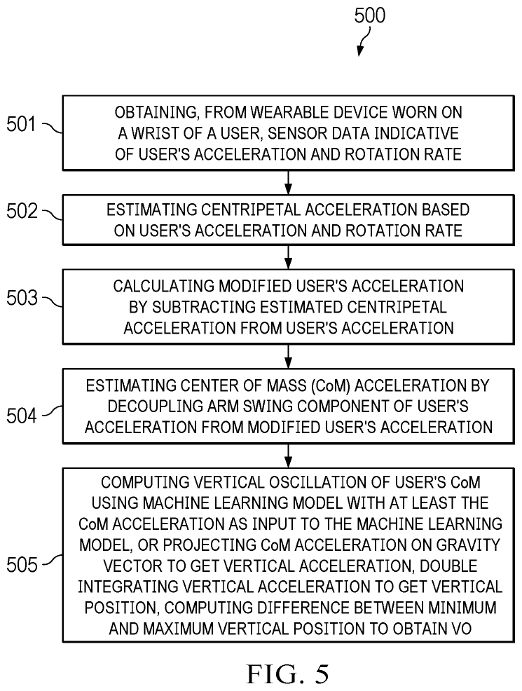

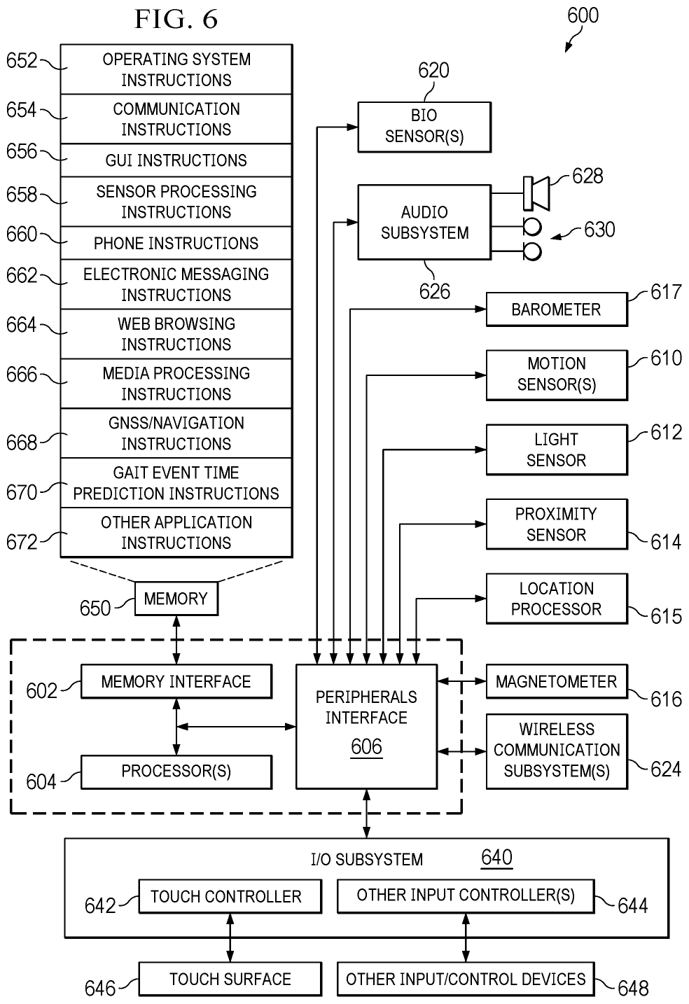

FIG. 5 is a flow diagram of process 500 for estimating VO at the wrist, according to some embodiments. Process 500 can be implemented by, for example, using system architecture 600 described in reference to FIG. 6.

Process 500 includes the steps of: obtaining, from a wearable device worn on a wrist of a user, sensor data indicative of the user's acceleration and rotation rate (501); estimating centripetal acceleration based on the user's acceleration and rotation rate (502); calculating a modified user's acceleration by subtracting the estimated centripetal acceleration from the user's acceleration (504); estimating center of mass (CoM) acceleration by decoupling an arm swing component of the user's acceleration from the modified user's acceleration (505); and computing vertical oscillation of the user's CoM using a machine learning model with at least the CoM acceleration as input to the machine learning mode, or projecting the CoM acceleration on a gravity vector to get vertical acceleration, double integrating the vertical acceleration to get vertical position, computing the difference between maximum and minimum vertical positions per step to obtain VO (506).

### Exemplary System Architectures

FIG. 6 illustrates example system architecture 600 implementing the features and operations described in reference to FIGS. 1-5. Architecture 600 can include memory interface 602, one or more hardware data processors, image processors and/or processors 604 and peripherals interface 606. Memory interface 602, one or more processors 604 and/or peripherals interface 606 can be separate components or can be integrated in one or more integrated circuits. System architecture 600 can be included in any suitable electronic device, including but not limited to: a smartphone, smartwatch, tablet computer, fitness band, laptop computer and the like.

Sensors, devices and subsystems can be coupled to peripherals interface 606 to provide multiple functionalities. For example, one or more motion sensors 610, light sensor 612 and proximity sensor 614 can be coupled to peripherals interface 606 to facilitate motion sensing (e.g., acceleration, rotation rates), lighting and proximity functions of the mobile device. Location processor 615 can be connected to peripherals interface 606 to provide geo-positioning. In some implementations, location processor 615 can be a GNSS receiver, such as the Global Positioning System (GPS) receiver. Electronic magnetometer 616 (e.g., an integrated circuit chip) can also be connected to peripherals interface 606 to provide data that can be used to determine the direction of magnetic North. Electronic magnetometer 616 can provide data to an electronic compass application. Motion sensor(s) 610 can include one or more accelerometers and/or gyros configured to determine change of speed and direction of movement. Barometer 617 can be configured to measure atmospheric pressure. Biosensors 320 can include a heart rate sensor, such as a photoplethysmography (PPG) sensor, electrocardiography (ECG) sensor, etc.

Communication functions can be facilitated through wireless communication subsystems 624, which can include radio frequency (RF) receivers and transmitters (or transceivers) and/or optical (e.g., infrared) receivers and transmitters. The specific design and implementation of the communication subsystem 624 can depend on the communication network(s) over which a mobile device is intended to operate. For example, architecture 600 can include communication subsystems 624 designed to operate over a GSM network, a GPRS network, an EDGE network, a Wi-Fi™ network and a Bluetooth™ network. In particular, the wireless communication subsystems 624 can include hosting protocols, such that the mobile device can be configured as a base station for other wireless devices.

Audio subsystem 626 can be coupled to a speaker 628 and a microphone 630 to facilitate voice-enabled functions, such as voice recognition, voice replication, digital recording and telephony functions. Audio subsystem 626 can be configured to receive voice commands from the user.

I/O subsystem 640 can include touch surface controller 642 and/or other input controller(s) 644. Touch surface controller 642 can be coupled to a touch surface 646. Touch surface 646 and touch surface controller 642 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 646. Touch surface 646 can include, for example, a touch screen or the digital crown of a smart watch. I/O subsystem 640 can include a haptic engine or device for providing haptic feedback (e.g., vibration) in response to commands from processor 604. In an embodiment, touch surface 646 can be a pressure-sensitive surface.

Other input controller(s) 644 can be coupled to other input/control devices 648, such as one or more buttons, rocker switches, thumb-wheel, infrared port and USB port. The one or more buttons (not shown) can include an up/down button for volume control of speaker 628 and/or microphone 640. Touch surface 646 or other controllers 644 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 646; and a pressing of the button for a second duration that is longer than the first duration may turn power to the mobile device on or off. The user may be able to customize a functionality of one or more of the buttons. The touch surface 646 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 602 can be coupled to memory 650. Memory 650 can include high-speed random access memory and/or non-volatile memory, such as one or more magnetic disk storage devices, one or more optical storage devices and/or flash memory (e.g., NAND, NOR). Memory 650 can store operating system 652, such as the iOS operating system developed by Apple Inc. of Cupertino, California. Operating system 652 may include instructions for handling basic system services and for performing hardware dependent tasks. In some implementations, operating system 652 can include a kernel (e.g., UNIX kernel).

Memory 650 may also store communication instructions 654 to facilitate communicating with one or more additional devices, one or more computers and/or one or more servers, such as, for example, instructions for implementing a software stack for wired or wireless communications with other devices, such as a sleep/wake tracking device. Memory 650 may include graphical user interface instructions 656 to facilitate graphic user interface processing; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GNSS/Location instructions 668 to facilitate generic GNSS and location-related processes and instructions; and gait event time prediction instructions 670 that implement the features and processes described in reference to FIGS. 1 and 2. Memory 650 further includes application instructions 672 for performing various applications that utilize VO (e.g., fitness applications, health monitoring applications).

Each of the above identified instructions and applications can correspond to a set of instructions for performing one or more functions described above. These instructions need not be implemented as separate software programs, procedures, or modules. Memory 650 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
obtaining, with at least one processor of a wearable device worn on a wrist of a user, sensor data indicative of the user's acceleration and rotation rate;
estimating, with the at least one processor, centripetal acceleration based on the user's acceleration and rotation rate;
calculating, with the at least one processor, a modified user's acceleration by subtracting the estimated centripetal acceleration from the user's acceleration;
estimating, with the at least one processor, center of mass (CoM) acceleration by decoupling an arm swing component of the user's acceleration from the modified user's acceleration; and
computing, with the at least one processor, vertical oscillation of the user's CoM using a machine learning model with at least the CoM acceleration as input to the machine learning model.

2. The method of claim 1, where estimating centripetal acceleration comprises:
determining a primary rotational axis based on the user's rotation rate;
estimating a tangential acceleration based on the user's acceleration and step frequency;
estimating tangential velocity by integrating the tangential acceleration;
calculating a magnitude of the arm swing angular rotation rate about the primary rotational axis; and
estimating the centripetal acceleration as a cross-product of the magnitude of the arm swing rotation rate and the tangential velocity.

3. The method of claim 2, further comprising:
computing, with the at least one processor, a vertical oscillation of the user's CoM using the machine learning model with the CoM acceleration, tangential acceleration, estimated centripetal acceleration, user acceleration, user rotation rate and primary rotational axis as inputs to the machine learning model.

4. The method of claim 1, wherein the primary rotational axis is determined using principal component analysis (PCA).

5. The method of claim 1, wherein estimating a tangential acceleration includes filtering the tangential acceleration from the user's acceleration based on a step frequency of the user.

6. The method of claim 1, wherein decoupling the arm swing component of the user's acceleration from the modified user's acceleration includes filtering the modified user's acceleration based on a step frequency of the user.

7. The method of claim 1, wherein the machine learning model is a random forest that outputs an estimate of vertical oscillation at the CoM per stride.

8. A method comprising:
obtaining, with at least one processor of a wearable device worn on a wrist of a user, sensor data indicative of the user's acceleration and rotation rate;
estimating, with the at least one processor, centripetal acceleration based on the user's acceleration and rotation rate;
calculating, with the at least one processor, a modified user's acceleration by subtracting the estimated centripetal acceleration from the user's acceleration;
estimating, with the at least one processor, center of mass (CoM) acceleration by decoupling an arm swing component of the user's acceleration from the modified user's acceleration;
determining, with the at least one processor, a gravity vector based on the sensor data;
determining, with the at least one processor, vertical acceleration by projecting the CoM acceleration onto the gravity vector;
integrating, with the at least one processor, the vertical acceleration to get vertical velocity;
integrating, with the at least one processor, the vertical velocity to get vertical position;
computing, with the at least one processor, vertical oscillation as the difference between maximum and minimum vertical position per step.

9. A system comprising:
at least one processor;
memory storing instructions that when executed by the at least one processor, cause the at least one processor to perform operations comprising:
obtaining sensor data indicative of the user's acceleration and rotation rate;
estimating centripetal acceleration based on the user's acceleration and rotation rate;
calculating a modified user's acceleration by subtracting the estimated centripetal acceleration from the user's acceleration;
estimating center of mass (CoM) acceleration by decoupling an arm swing component of the user's acceleration from the modified user's acceleration; and
computing vertical oscillation of the user's CoM using a machine learning model with at least the CoM acceleration as input to the machine learning model.

10. The system of claim 9, where estimating centripetal acceleration comprises:
determining a primary rotational axis based on the user's rotation rate;
estimating a tangential acceleration based on the user's acceleration and step frequency;
estimating tangential velocity by integrating the tangential acceleration;
calculating a magnitude of the arm swing angular rotation rate about the primary rotational axis; and
estimating the centripetal acceleration as a cross-product of the magnitude of the arm swing rotation rate and the tangential velocity.

11. The system of claim 10, further comprising:
Computing a vertical oscillation of the user's CoM using the machine learning model with the CoM acceleration, tangential acceleration, estimated centripetal acceleration, user acceleration, user rotation rate and primary rotational axis as inputs to the machine learning model.

12. The system of claim 9, wherein the primary rotational axis is determined using principal component analysis (PCA).

13. The system of claim 9, wherein estimating a tangential acceleration includes filtering the tangential acceleration from the user's acceleration based on a step frequency of the user.

14. The system of claim 9, wherein decoupling the arm swing component of the user's acceleration from the modified user's acceleration includes filtering the modified user's acceleration based on a step frequency of the user.

15. The system of claim 9, wherein the machine learning model is a random forest that outputs an estimate of vertical oscillation at the CoM per stride.

16. A system comprising:
obtaining sensor data indicative of the user's acceleration and rotation rate;
estimating centripetal acceleration based on the user's acceleration and rotation rate;
calculating a modified user's acceleration by subtracting the estimated centripetal acceleration from the user's acceleration;
estimating center of mass (CoM) acceleration by decoupling an arm swing component of the user's acceleration from the modified user's acceleration;
determining a gravity vector based on the sensor data;
determining vertical acceleration by projecting the CoM acceleration onto the gravity vector;
integrating the vertical acceleration to get vertical velocity;
integrating the vertical velocity to get vertical position;
computing vertical oscillation as the difference between maximum and minimum vertical position per step.

17. A non-transitory, computer-readable storage medium having stored thereon instructions that when executed by the at least one processor, causes the at least one processor to perform operations comprising:
obtaining sensor data indicative of the user's acceleration and rotation rate;
estimating centripetal acceleration based on the user's acceleration and rotation rate;
calculating a modified user's acceleration by subtracting the estimated centripetal acceleration from the user's acceleration;
estimating center of mass (CoM) acceleration by decoupling an arm swing component of the user's acceleration from the modified user's acceleration; and
computing vertical oscillation of the user's CoM using a machine learning model with at least the CoM acceleration as input to the machine learning model.

18. The non-transitory, computer-readable storage medium of claim 17, where estimating centripetal acceleration comprises:
determining a primary rotational axis based on the user's rotation rate;
estimating a tangential acceleration based on the user's acceleration and step frequency;
estimating tangential velocity by integrating the tangential acceleration;
calculating a magnitude of the arm swing angular rotation rate about the primary rotational axis; and
estimating the centripetal acceleration as a cross-product of the magnitude of the arm swing rotation rate and the tangential velocity.

19. The non-transitory, computer-readable storage medium of claim 18, further comprising:
computing a vertical oscillation of the user's CoM using the machine learning model with the CoM acceleration, tangential acceleration, estimated centripetal acceleration, user acceleration, user rotation rate and primary rotational axis as inputs to the machine learning model.

20. The non-transitory, computer-readable storage medium of claim 17, wherein the primary rotational axis is determined using principal component analysis (PCA).
